(19) United States
(12) Patent Application Publication
(10) Pub. No.: US 2004/0172040 A1
(43) Pub. Date: Sep. 2, 2004

(54) Bone compression devices and systems and methods of contouring and using same

(76) Inventor: Michael H. Heggeness, Bellaire, TX (US)

Correspondence Address: ANTHONY F. MATHENY, ANDREWS & KURTH L.L.P., 600 TRAVIS, SUITE 4200, HOUSTON, TX, 77002, US

(21) Appl. No.: 10/779,980

(22) Filed: Feb. 17, 2004

Related U.S. Application Data
(63) Continuation-in-part of application No. 10/273,853, filed on Oct. 17, 2002.
(60) Provisional application No. 60/344,980, filed on Oct. 19, 2001.

(51) Int. Cl. A61B 17/58

(52) U.S. Cl. 606/105

(57) ABSTRACT

The invention relates to bone compression devices and bone compression systems, and in particular, to bone compression devices and systems for use in connection with vertebrae. The bone compression devices and bone compression systems are disposed, or installed, along at least one bone to maintain the at least one bone in a desired spatial relationship. Broadly, the invention is directed to a bone compression device for placing in communication with at least one bone having a bone radius of curvature, the bone compression device comprising a plate having a pre-formed shape, a deformed shape, and at least one elastic shape therebetween, the pre-formed shape having a pre-formed radius of curvature less than the bone radius of curvature, the deformed shape having a deformed radius of curvature greater than the bone radius of curvature, and at least one of the at least one elastic shapes having an elastic radius of curvature that substantially corresponds to the bone radius of curvature. The invention is also directed to bone compression systems having a bone compression device and a tensioner for facilitating the movement of the plate from the pre-formed shape to the at least one elastic shape. Methods of maintaining a bone in a spatial relationship and methods of contouring the bone compression devices are also disclosed.

### RELATED APPLICATION

This application is a continuation-in-part of U.S. Non-Provisional patent application Ser. No. 10/273,853, filed Oct. 17, 2002, which claims the benefit of U.S. Provisional Patent Application Serial No. 60/344,980, filed Oct. 19, 2001.

### BACKGROUND OF THE INVENTION

1. Field Of The Invention

This invention relates to surgical devices, and in particular, to bone compression devices for maintaining bones, namely, one or more vertebrae, in a desired spatial relationship. The invention also relates to methods for maintaining at least one bone in a spatial relationship and methods for contouring the bone compression devices for use in maintaining bones in a desired spatial relationship.

2. Description Of Related Art

The use of bone compression devices in connection with vertebrae are known in the art. Many of these prior bone compression devices are directed to fusing together two or more vertebrae. However, the success rate offusing together four or more levels is extremely low, i.e., approximately 50% as compared to approximately 95% for two level fusion and 98%-99% for single level fusion. At least one reason contemplated for the increase in the percentage of failures of the bone compression devices is that the bone compression devices do not substantially correspond to the anatomical curvature of the bone to which they are applied. Accordingly, prior to installation, or implantation by the surgeon, the bone compression devices must be manipulated or shaped to substantially correspond to the shape of the bone or bones. As the length of the bone compression device increases, e.g., to fuse three or more vertebrae, the amount of manipulation, e.g., bending, required generally increases. As a result of this manipulation, the bone compression device may become permanently deformed, and thus weakened, or experience hysteresis.

As with most all materials used to form surgical implants and devices, e.g., titanium and stainless steel and various alloys, the devices include a pre-formed shape, i.e., the shape of the device as formed during manufacture, and a deformed shape, i.e., the shape of the device after sufficient force is exerted on the device to permanently change the shape of the device. In between the preformed shape and the deformed shape are numerous elastic shapes. These elastic shapes have a tendency to revert back toward the pre-formed shape over a period time ranging from near immediacy, e.g., a few seconds, to a number of weeks or even months. In fact, most materials experience a certain amount of reversion of shape immediately and then, over an extended period of time, experience additional amounts of reversion of shape. This reversion of shape is referred to as hysteresis. One example of hysteresis is illustrated in materials having what has been referred to as “metal memory.” Because of hysteresis, many prior bone compression devices do not remain properly shaped and ultimately fail.

Both the weakening of the bone compression device and hysteresis is further complicated by the use of fasteners, e.g., bone screws or bolts, to secure the bone compression devise to the bone. If the fastener holes are misshapen during the manipulation by the surgeon, the fasteners, when installed, generally force the bone compression device back to its original shape, i.e., away from manipulated shape formed by the surgeon to correspond to the shape of the bone.

For example, in one prior approach, the bone compression device is generally straight. Because the spine is lordotic, the bone compression device must be manipulated, or bent, by the surgeon to attempt to shape the bone compression device to correspond to the curvature of the spine. These devices, after implantation, experience hysteresis resulting in the bone compression device attempting to revert back to its pre-formed shape, i.e., generally straight. As a result, the bone compression device experiences a higher incidence of failure over long lengths. Such hysteresis is increased by the installation of the fasteners into misshapen fastener holes to secure the bone compression device to the bone. As bone screws or other fasteners are inserted to secure the bone compression device to the bone, pressure is placed on the plate to push it onto the bone resulting in the plate straightening out, i.e., being manipulated away from the shape desired by the surgeon.

In another prior approach, the bone compression device is slightly contoured to approximate the curvature of the bone to which the bone compression device is to be connected. However, the pre-formed curvature of the bone compression device rarely, if ever, accurately corresponds to the curvature of the bone to which it is to be implanted. Therefore, the surgeon must still manipulate these bone compression devices to provide additional lordosis or curvature to correspond to the curvature of the bone. While the amount of manipulation by the surgeon may be lessened, these bone compression devices also experience hysteresis resulting in potential failure of the bone compression device.

Accordingly, prior to the development of the present invention, there has been no tensioners, bone compressions devices, or bone compression systems, which: decrease the rate of failure of the bone compression devices due to hysteresis; utilize hysteresis to increase the rate of success of the bone compression devices; decrease the rate of failure of the bone compression devices when employed on long bones or multiple bones, e.g., three or more vertebrae; and provide compressive forces to the bone to which the bone compression devices are implanted, thereby increasing the grip of the bone compression device on the bone. Therefore, the art has sought tensioners, bone compressions devices, and bone compression systems, which: decrease the rate of failure of the bone compression devices due to hysteresis; utilize hysteresis to increase the rate of success of the bone compression devices; decrease the rate of failure of the bone compression devices when employed on long bones or multiple bones, e.g., three or more vertebrae; and provide compressive forces to the bone to which the bone compression devices are implanted, thereby increasing the grip of the bone compression device on the bone. It is believed that the present invention will achieve these objectives and overcome the disadvantages of other compression devices and bone compression systems for placing in communication with at least one bone having a bone radius of curvature, methods of maintaining at least two vertebrae in a spatial relationship with each other, and methods of contouring bone compression devices in the field of the invention, but its results or effects are still dependent upon the skill and training of the operators and surgeons.

### SUMMARY OF INVENTION

In accordance with the invention, the foregoing advantages have been achieved through the present tensioner for facilitating the movement of a plate of a bone compression device, the plate having a pre-formed shape and at least one elastic shape that substantially corresponds to at least one bone radius of curvature of at least one bone, the tensioner comprising: a shaft, a base, and at least two arms in communication with the shaft, the at least two arms adapted to be releasably secured to the plate, the shaft having a shaft first end, a shaft second end, a shaft longitudinal axis, and at least one screw groove disposed along the shaft longitudinal axis to facilitate the movement of the at least two arms along the longitudinal axis of the shaft, the shaft second end being connected to the base.

A further feature of the tensioner is that the tensioner may further comprise at least three arms adapted to be releasably secured to the plate. An additional feature of the tensioner is that the tensioner may further comprise at least four arms adapted to be releasably secured to the plate. Another feature of the tensioner is that at least two of the at least two arms of the tensioner may include an arm end, the arm end having a shape that permits insertion of the arm end into a slot disposed along the plate. Still another feature of the tensioner is that at least two of the at least two arms of the tensioner may include an arm end, the arm end having a hook. A further feature of the tensioner is that at least two of the at least two arms of the tensioner may include an arm end, the arm end having a spatula member. An additional feature of the tensioner is that at least two of the at least two arms of the tensioner may be operatively associated with an arm connector by a hinge member. Another feature of the tensioner is that the shaft second end may be rotatably connected to the base such that the shaft is permitted to rotate and the base is permitted to remain stationary. Still another feature of the tensioner is that the shaft of the tensioner may be rotatably connected to the base by a ball joint. A further feature of the tensioner is that the tensioner may be rotatably connected to the base by a base shaft disposed within a cavity of the shaft.

In accordance with the invention, the foregoing advantages have also been achieved through the present tensioner for facilitating the movement of a plate of a bone compression device, the plate having a pre-formed shape and at least one elastic shape that substantially corresponds to at least one bone radius of curvature of at least one bone, the tensioner comprising: a splint having a first end and a second end and at least two fastening members, wherein at least one of the at least two fastening members is adapted to be releasably secured to the splint.

A further feature of the tensioner is that each of the at least two fastening members is adapted to be releasably secured to the splint.

In accordance with the invention, the foregoing advantages have also been achieved through the present tensioner for facilitating the movement of a plate of a bone compression device, the plate having a pre-formed shape and at least one elastic shape that substantially corresponds to at least one bone radius of curvature of at least one bone, the tensioner comprising: a shaft, a base, and at least two arms in communication with the shaft, the at least two arms adapted to be releasably secured to the plate, the shaft having a shaft first end, a shaft second end, a shaft longitudinal axis, an inner shaft member slidably engaged with an outer shaft member, and at least one arm connector support member disposed along the shaft longitudinal axis to maintain the at least two arms at a position along the longitudinal axis of the shaft, the shaft second end being connected to the base.

A further feature of the tensioner is that the shaft outer member may include a shaft flange. Another feature of the tensioner is that each of the at least one arm connector support members may be movable support members and the inner shaft member may include at least one inner shaft aperture corresponding to each of the at least one movable support members.

A further feature of the tensioner is that the base may be rotatably connected to the base such that the shaft is permitted to rotate and the base is permitted to remain stationary.

In accordance with the invention, the foregoing advantages have also been achieved through the present bone compression device for placing in communication with at least one bone having at least one bone radius of curvature, the bone compression device comprising a plate having a preformed shape, the pre-formed shape having at least one pre-formed radius of curvature corresponding to each of the at least one bone radii of curvature, each of the at least one pre-formed radii of curvature being less than each of the corresponding at least one bone radii of curvature.

A further feature of the bone compression device is that the plate may include a deformed shape and at least one elastic shape between the pre-formed shape and the deformed shape, the deformed shape having a deformed radius of curvature greater than at least one of the bone radii of curvature, and at least one of the at least one elastic shapes having an elastic radius of curvature that substantially corresponds to at least one of the at least one bone radii of curvature. An additional feature of the bone compression device is that the plate may include a first end, a second end, and at least two attachment members. Another feature of the bone compression device is that at least one of the at least two attachment members may be a hole. Still another feature of the bone compression device is that at least one of the at least two attachment members may be a loop. A further feature of the bone compression device is that at least one of the two attachment members may be disposed at the first end of the plate and another of the at least two attachment members is disposed at the second end of the plate. An additional feature of the bone compression device is that at least one of the at least two attachment members may be a hole. Another feature of the bone compression device is that at least one of the at least two attachment members may be a loop. Still another feature of the bone compression device is that the plate may include a first end, a second end, and at least two attachment members. A further feature of the bone compression device is that the plate may include a length having a longitudinal axis and a width having a lateral axis, at least one of the at least one pre-formed radii of curvature being disposed along a portion of the length. An additional feature of the bone compression device is that the plate may include a length having a longitudinal axis and a width having a lateral axis, at least one of the at least one pre-formed radii of curvature being disposed along a portion of the width. Another feature of the bone compression device is that the plate may include a first end, a second end, at least two fastener holes disposed near the first end, and at least two fastener holes disposed near the second end.

In accordance with the invention, the foregoing advantages have also been achieved through the present bone compression system for placing in communication with at least one bone having at least one bone radius of curvature, the bone compression system comprising: aplate having a first end, a second end, a pre-formed shape, a deformed shape, and at least one elastic shape between the pre-formed shape and the deformed shape, the pre-formed shape having at least one pre-formed radius of curvature wherein at least one of the at least one pre-formed radii of curvature is less than at least one of the at least one bone radii of curvature, the deformed shape having a deformed radius of curvature greater than at least one of the at least one bone radii of curvature, and at least one of the at least one elastic shapes having an elastic radius of curvature that substantially corresponds to at least one of the at least one bone radii of curvature; and a tensioner for facilitating the movement of the plate from the pre-formed shape to the at least one elastic shape that substantially corresponds to at least one of the at least one bone radii of curvature, the tensioner including a shaft, a base, and at least two arms adapted to be releasably secured to the plate, the shaft having a shaft first end, a shaft second end, a shaft longitudinal axis, and at least one screw groove disposed along the shaft longitudinal axis to facilitate the movement of the at least two arms along the longitudinal axis of the shaft, the shaft second end being connected to the base.

A further feature of the bone compression system is that the first and second attachment members of the plate may be slots, the first slot being disposed at the first end of the plate and the second slot being disposed at the second end of the plate. An additional feature of the bone compression system is that at least two of the at least two arms of the tensioner may include an arm end, the arm end having a shape that permits insertion of the arm ends into the first and second slots. Another feature of the bone compression system is that the first and second attachment members of the plate may be loops, the first loop being disposed at the first end of the plate and the second loop being disposed at the second end of the plate. Still another feature of the bone compression system is that at least two of the at least two arms of the tensioner may include an arm end, the arm end having a hook that permits insertion of the arm end into the first and second loops. A further feature of the bone compression system is that at least two of the at least two arms of the tensioner may include an arm end, the arm end having a spatula member. An additional feature of the bone compression system is that at least two of the at least two arms of the tensioner may be operatively associated with an arm connector by a hinge member. Another feature of the tensioner is that the shaft second end may be rotatably connected to the base such that the shaft is permitted to rotate and the base is permitted to remain stationary. Still another feature of the bone compression system is that the shaft of the tensioner may be rotatably connected to the base by a ball joint. An further feature of the bone compression system is that the shaft of the tensioner may be rotatably connected to the base by a base shaft disposed within a cavity of the shaft. An additional feature of the bone compression system is that the plate may include a length having a longitudinal axis and a width having a lateral axis, the pre-formed radius of curvature and the at least one elastic radius of curvature being disposed along the length. Another of the bone compression system is that the plate may include a length having a longitudinal axis and a width having a lateral axis, the pre-formed radius of curvature and the at least one elastic radius of curvature being disposed along the width.

The tensioners, bone compressions devices, and bone compression systems of the invention have the advantages of: decreasing the rate of failure of the bone compression devices due to hysteresis; utilize hysteresis to increasing the rate of success of the bone compression devices; decreasing the rate of failure of the bone compression devices when employed on long bones or multiple bones, e.g., three or more vertebrae; and providing compressive forces to the bone to which the bone compression devices are implanted, thereby increasing the grip of the bone compression device on the bone. As mentioned above, it is believed that the present invention will achieve these objectives and overcome the disadvantages of other surgical devices and surgical systems and methods in the field of the invention, but its results or effects are still dependent upon the skill and training of the operators and surgeons.

### BRIEF DESCRIPTION OF DRAWINGS

FIG. 1A is a longitudinal side view of a human spinal column.

FIG. 1B is a detailed side view of two human vertebrae having a disk disposed between the two vertebrae.

FIG. 1C is a detailed side view of two human vertebrae with a bone graft disposed between two vertebrae.

FIG. 2 is a schematic showing the radius of curvature of a longitudinal curve.

FIG. 3A is a longitudinal side view of a prior art bone compression device before being formed into its implantation shape.

FIG. 3B is a longitudinal side view of the prior art bone compression device shown in FIG. 3A in its implantation shape.

FIG. 4 is a longitudinal side view of a specific embodiment of the bone compression device of the present invention.

FIG. 5 is a top view of the bone compression device shown in FIG. 4.

FIG. 6 is a longitudinal side view of another specific embodiment of the bone compression device of the present invention.

FIG. 7 is a top view of the bone compression device shown in FIG. 6.

FIG. 8 is a side view of a specific embodiment of the tensioner of one specific embodiment of the bone compression system of the present invention.

FIG. 9 is a side view of a specific embodiment of the string of one specific embodiment of the bone compression system of the present invention.

FIG. 10 is side view of one specific embodiment of the bone compression system of the present invention.

FIG. 11 is a side view of another specific embodiment of the bone compression system of the present invention.

FIG. 12 is a partial cross-sectional view of the shaft and base of one specific embodiment of the tensioner of the present invention.

FIG. 13 is a partial cross-sectional view of the shaft and base of another specific embodiment of the tensioner of the present invention.

FIG. 14 is a partial side view of the arm end of one specific embodiment of the tensioner of the present invention.

FIG. 15 is a partial side view of the arm end of another specific embodiment of the tensioner of the present invention.

FIG. 16 is a partial side view of the arm end of still another specific embodiment of the tensioner of the present invention.

FIG. 17 is a side view of another specific embodiment of the bone compression system of the present invention.

FIG. 17 is a partial cross-sectional view of shaft of the embodiment shown in FIG. 17.

FIG. 19 is a side view of another specific embodiment of the bone compression system of the present invention having a bone compression device in its preformed shape.

FIG. 20 is a side view of the specific embodiment of the bone compression system shown in FIG. 19 having a bone compression device in one of its elastic shapes.

While the invention will be described in connection with the preferred embodiment, it will be understood that it is not intended to limit the invention to that embodiment. On the contrary, it is intended to cover all alternatives, modifications, and equivalents, as may be included within the spirit and scope of the invention as defined by the appended claims.

### DETAILED DESCRIPTION AND SPECIFIC EMBODIMENTS

The present invention is directed to bone compression devices and bone compression systems for maintaining at least one bone in a desired spatial relationship. While the description of the bone compression devices, bone compression systems, and methods of contouring the bone compression devices will be directed to use in connection with two or more vertebrae, it is to be understood that the bone compression devices, bone compression systems, methods of maintaining at least two vertebrae in a spatial relationship with each other, and methods of contouring the bone compression devices of the invention may be used, or- performed, in connection with any bone in which it is desired to maintain, or place, at least one bone in a desired spatial relationship, e.g., the pelvis, the femur, the fibula, the tibia, humerus, ulna, radius, or any other bone. For example, the bone compression devices of the invention maybe employed in long bone, e.g., femur, and pelvic fracture fixation. Further, the bone compression device may be used in connection with rigid locked screwplates and screw-plates that permit some screw subsidence or angulation, both of which are bone compression devices known in the art.

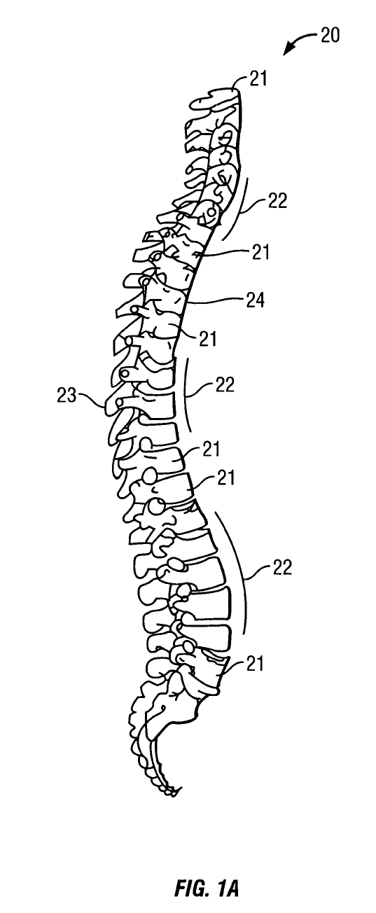

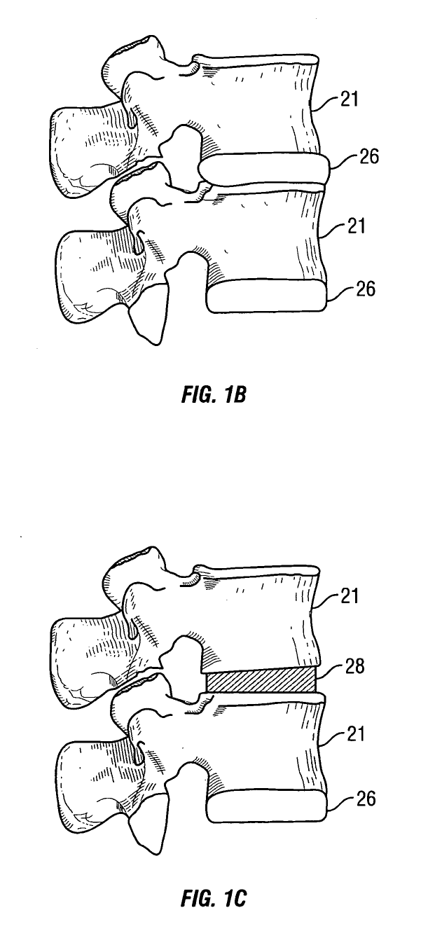

In the preferred embodiments, the bone compression devices are utilized to maintain one or more vertebrae, and more preferably, three or more vertebrae, of the spine, and in particular, the cervical vertebrae of humans, in a desired spatial relationship. In these embodiments, prior to the installation of the bone compression device, one or more bone grafts are generally disposed between two or more vertebrae. As illustrated in FIGS. 1B and 1C, for example, after a disk 26, located between two vertebrae 21, is removed from spine 20, a bone graft 28, e.g., a portion of the patients' own bone, an allograft (portion of bone donated from another individual), or synthetic bone grafts or cages or boxes such as those made from carbon fiber, metal (particularly titanium), or ceramics, is usually disposed in the space created by the removal of the disk 26. Thereafter, the bone compression devices of the invention are installed and secured to at least the vertebra 21 disposed above the graft and the vertebra 21 disposed below the graft. Accordingly, bone graft 28 is compressed between the two vertebrae 21 to maintain bone graft 28 in a spatial relationship with vertebrae 21.

It is contemplated that the bone compression devices of the invention are suitable for fusing, e.g., being secured to, more than three vertebrae having one or more grafts disposed between two or more of the vertebrae. Therefore, in these embodiments, the vertebrae in proximity to the removed disk(s) are maintained in a desired spatial relationship to one another. Therefore, the vertebrae are permitted to heal, and, in some cases, the synthetic bone graft is permitted to be incorporated into the vertebrae using bone growth factors and other biologically active substances to facilitate the growth of bone over the synthetic bone graft.

Referring now to FIGS. 1A, spine 20 includes vertebrae 21 and has posterior side 23 and anterior side 24. Spine 20 includes numerous bone radius of curvatures 22 along the longitudinal length of spine 20. As shown in FIG. 1, bone radius of curvatures 22 are identified along anterior side 24 of spine 20. Although the bone compression devices of the invention may be installed, or implanted, along the posterior side 23 of spine 20, generally, bone compression devices are implanted along anterior side 24 of spine 20.

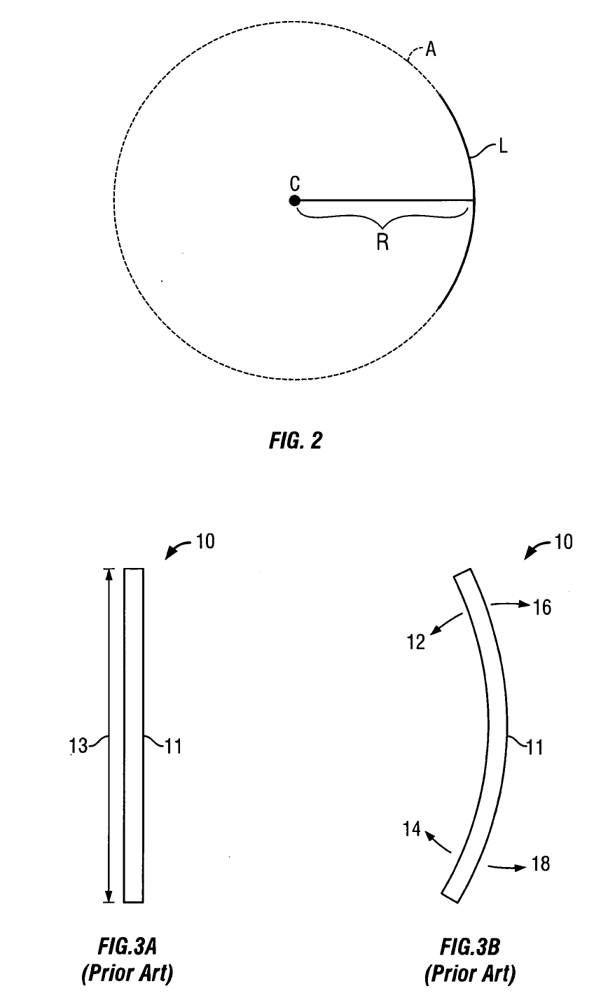

Radius of curvature, as used herein referring to the shape of the bone(s) as well as the shape of the bone compression devices and systems, is measured by determining the radius of a circle formed by the longitudinal curve of the bone (bone radius of curvature) or bone compression device (pre-formed radius of curvature, deformed radius ofcurvature, and elastic radius of curvature) placed along the circumference of the circle formed by the longitudinal curve of the bone or bone compression device. Accordingly, the larger the circle formed by the longitudinal curve of the bone or bone compression device, the larger the corresponding curvature, i.e., the greater the radius of curvature. Therefore, as the longitudinal curve approaches a straight line, the radius of curvature approaches infinity. FIG. 2 illustrates how radius of curvature R is determined along curvature L with respect to circle A having center C.

As illustrated in FIGS. 3A and 3B, one prior bone compression device 10 includes plate 11 having longitudinal length 13. As shown in FIG. 3A, plate is generally straight. As such, it must be manipulated, e.g., bent, to correspond to the shape of the bone to which it will be implanted. In other words, bone compression device 10 must be manipulated to correspond to the bone radius of curvature. As shown in FIG. 3B, plate 10 is bent in the direction of arrows 12 and 14 to create the desired radius of curvature. However, due to hysteresis, after plate 11 is bent in this manner plate 11 begins to revert back to its original shape (FIG. 3A) by moving in the direction of arrows 16 and 18. Alternatively, plate 11 is bent to its deformed shape, thereby weakening plate 11.

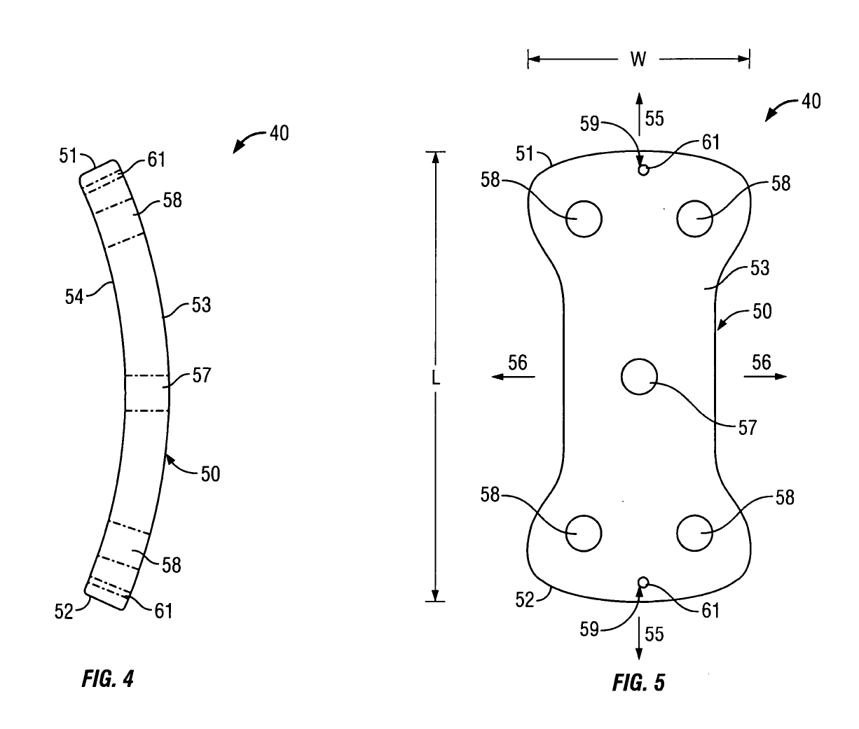

Referring now to FIGS. 4-7, in one aspect the present invention is directed to bone compression device 40 having plate 50, first end 51, second end 52, upper surface 53, lower surface 54, longitudinal axis 55 along length L of plate 50, and lateral axis 56 along width W of plate 50. Length L and Width W may have any measurement desired or necessary to secure bone compression device 40 the bone(s) desired to be compressed. For example, in embodiments in which three or more vertebrae are desired to be compressed, Length L must be of a distance sufficient to permit attachment of bone compression device 40 to each vertebrae. Therefore, length L of plate 50 will be greater in embodiments in which five vertebrae are to be compressed as compared to embodiments in which only two vertebrae are to be compressed.

Plate 50 also includes at least one fastener hole 58 for receiving fastener (not shown), e.g., bone screws, bolts, etc., to facilitate securing plate 50 to the bone. Preferably, each fastener hole is angled such that each fastener is placed through fastener holes 58 to be secured into the bone at an angle, thereby facilitating securing plate 50 to the bone. Additionally, multiple fastener holes 58 are disposed along the length L and width W of plate 50 as desired or necessary to facilitate securing plate 50 to the bone. For example, in embodiments in which three or more vertebrae are to be secured by bone compression device 40, plate 50 will preferably include fastener holes 58 such that at least one fastener will be inserted and secured to each of the vertebrae. Therefore, the total number of fastener holes 58 will be dependent upon the size of plate 50, the number of bones to be compressed, and the size of the bone(s) to be compressed.

Generally, plate 50 includes at least one fastener hole 58 disposed near first end 51 and at least one fastener hole 58 near second end 52. As shown in FIGS.4-7, plate 50 preferably includes at least two fastener holes 58 near first end 51 and two fastener holes 58 near second end 52. An advantage of this embodiment is that fasteners, e.g., bone screws, may be place through fastener holes 58 and secured into the bone, and thus securing plate 50 to the bone, in a manner that lessens the torsional resistance of plate 50. For example, a first fastener may be used to secure plate 50 to the bone by placing and securing the fastener through the upper right fastener hole 58 in FIG. 5. Next, a second fastener may be used to secure plate 50 to the bone by placing and securing the fastener through the lower left fastener hole 58 in FIG. 5. A third fastener may then be used to secure plate 50 to the bone by placing and securing the fastener through the upper left fastener hole 58 in FIG. 5. Thereafter, a fourth fastener may be used to secure plate 50 to the bone by placing and securing the fastener through the lower right fastener hole 58 in FIG. 5. All four fasteners may then be tightened as necessary to secure plate 50 to the bone.

Preferably, plate 50 includes plate interface, e.g., plate interface hole 57, for receiving a tool, e.g., tensioner 70 discussed in greater detail below, to facilitate handling of plate 50, moving plate 50 from its pre-formed shape to at least one of its at least one elastic shapes, and placement of plate 50 along the bone.

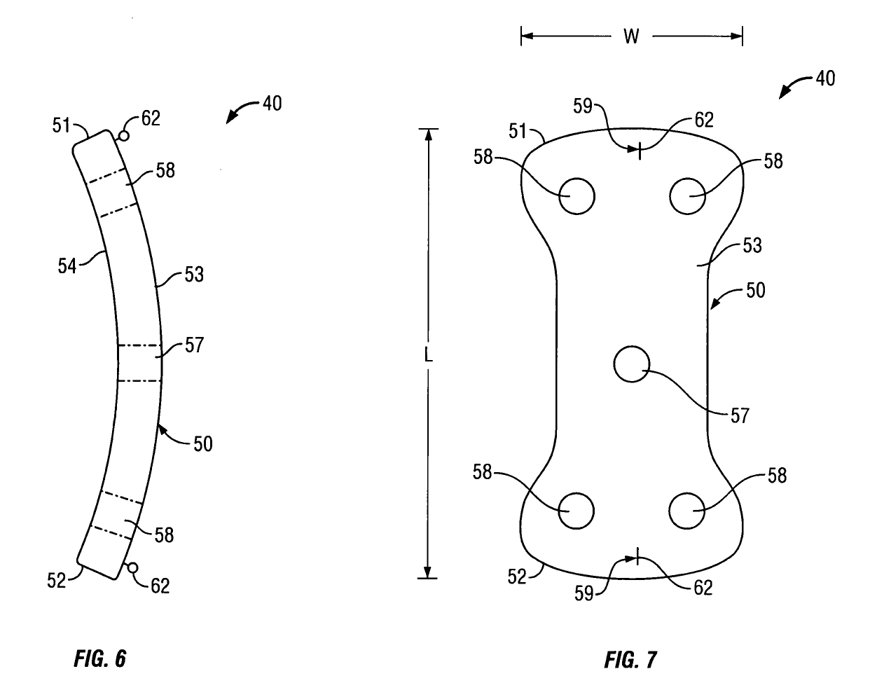

Pre-formed radius of curvature, deformed radius of curvature, and each of the at least one elastic radius of curvature therebetween are determined based upon the curvature of plate 50 along longitudinal axis 55 or lateral axis 56. As shown in FIGS. 4 and 6, plate 50 is in its pre-formed shape having pre-formed radius of curvature defined by the longitudinal curvature of lower surface 54.

Plate 50 preferably includes at least two attachment members 59. In the embodiment shown in FIGS. 4-5, each attachment member 59 is hole 61. In the embodiment shown in FIGS. 6-7, each attachment member 59 is loop 62.

While it is to be understood that the material from which bone compression device 50 is formed may be any material known to persons of ordinary skill in the art, the preferred material is titanium, titanium alloy, or other material having a relatively low coefficient of elasticity, thereby reducing the total number of elastic shapes bone compression device may take. As is readily apparent to those skilled in the art, the number of elastic shapes the bone compression device may take will depend on the material from which bone compression device is formed. Materials having a high coefficient of elasticity will have many more elastic shapes than a material having a low coefficient of elasticity. Suitable materials include titanium, titanium-vanadium-aluminum, cobaltchromium-molybdenum, as well as any other alloy, metal, or other material approved by the Food and Drug Administration.

Additionally, plate 50 may have any two or three dimensional shape, size, or thickness desired or necessary to be sufficiently secured to one or more bones.

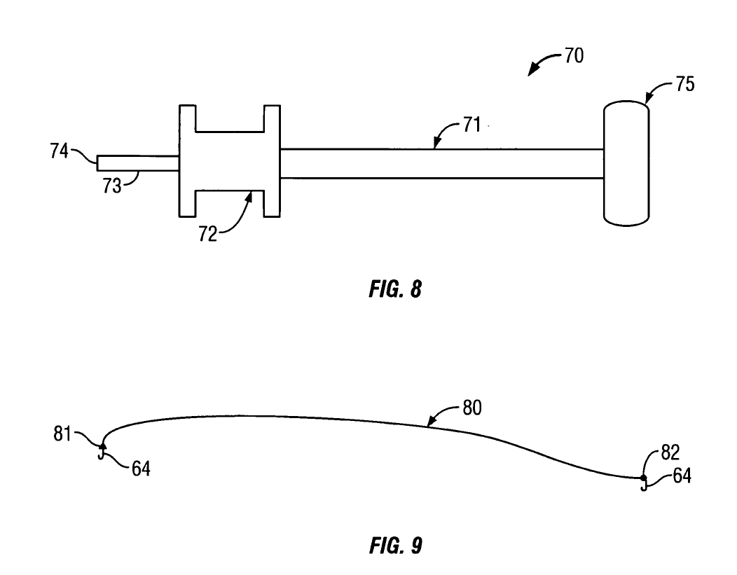

As discussed above, plate 50 preferably includes at least two attachment members, e.g., hole 61 or loop 62, for facilitating the contouring of plate 50, for example, by string 80 (FIG. 9) and tensioner 70. As shown in FIG. 8, tensioner 70 includes shaft 71 and spool 72. Spool 72 preferably includes a ratchet (not shown) to further facilitate winding string 80 around spool 72 and applying straightening forces to plate 50. Tensioner 70 also preferably includes at least one plate interface member 73 having interface member end 74 for interfacing with plate 50, e.g., by inserting at least one plate interface member 73 into at least one corresponding plate interface hole 57 disposed along plate 50, to further secure plate 50 to tensioner 70 thereby facilitating placement of plate 50 along the bone as well as tightening string 80 around spool 72. Interface member end 74 preferably is shaped to correspond with the shape of the plate interface. In one embodiment, plate interface hole 57 is a fastener hole 58. Tensioner 70 may also include handle 75 to facilitate gripping and turning tensioner 70 while tightening string 80 around spool 72.

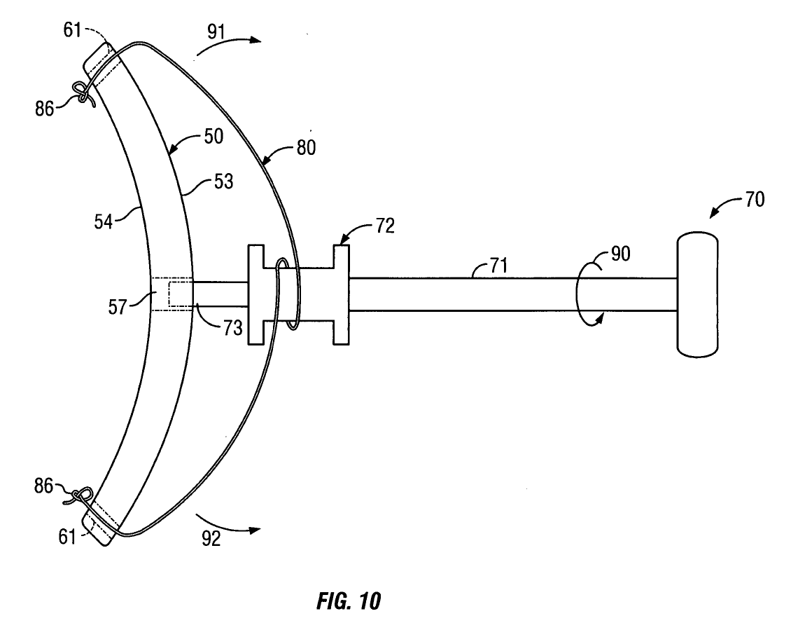

As illustrated in FIG. 9, string 80 includes first end 81 and second end 82 and may be releasably secured to plate 50 through any manner known to persons skilled inthe art. For example, string 80 may be passed through each attachment member 59 and tied in a knot 86 (FIG. 10). Therefore, after installation of plate 50, each knot may be cut to release plate 50. Alternatively, as shown in FIG. 8, first end 81 and second end 82 of string 80 may include hook 64 to be passed through each attachment member 59 to secure plate 50 during installation. Thereafter, each hook 64 may be removed from each attachment member 59 thereby releasing plate 50.

String 80 maybe formed out of any material known to persons skilled in the art provided that string 80 is strong enough to withstand the tensioning forces applied to string 80 during use without breaking. Preferred materials for forming string 80 include Kevlar and other polymers having high tensile strength.

Referring now to FIG. 10, tensioner 70 is placed in contact with plate 50 by inserting plate interface member 73 into plate interface hole 57. First end 81 of string 80 is passed through attachment member 59, i.e., hole 61 in FIG. 10, and tied into knot 86 to secure first end 81 to plate 50. Likewise, second end 82 of string 80 is passed through attachment member 59, i.e., hole 61 in FIG. 10, and tied into knot 86 to secure second end 82 to plate. String 80 is then wrapped around spool 72 of tensioner 70. Tensioner 70 may then be turned in the direction of arrow 90 to wind, or wrap, string 80 around spool 72. In doing so, string 80 exerts forces on plate 50 whereby place 50 is manipulated, or moved, in the direction of arrows 91 and 92 (straightening forces) to at least one of the elastic shapes of plate 50 until the elastic radius of curvature substantially corresponds to the bone radius of curvature, i.e., the implantation shape. Plate 50 may then be installed along one or more bones. After placement of plate 50 along one or more bones, tensioner 70 is turned in the opposite direction of arrow 90. Therefore, due to hysteresis, plate 50 will have a tendency to move in the opposite direction of arrows 91 and 92. In doing so, plate 50 will be further secured to the bone or bones.

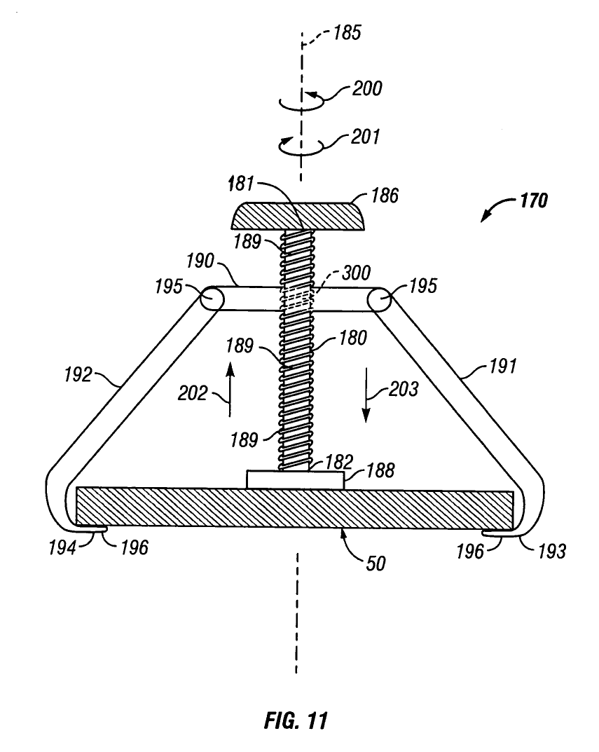

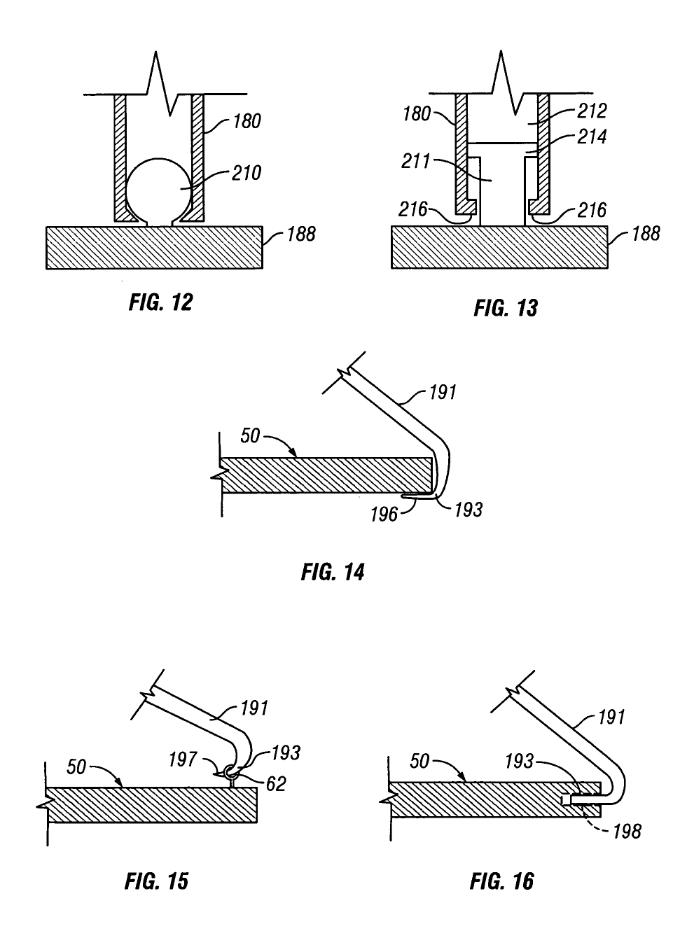

As illustrated in FIGS. 11-17, in other embodiments of the bone compression system, tensioner 170 comprises shaft 180 having first end 181, second end 182, shaft longitudinal axis 185, first arm 191 and second arm 192. First arm 191 and second arm 192 include arm ends 193 and 194 that are adapted to be releasably secured to plate 50. For example, arm end 193 and arm end 194 may include spatula member 196 (FIGS. 11, 14 and 17) or hooks 197 (FIG. 15) to be disposed through attachment members disposed along plate 50, e.g., or loops 62 (FIG. 15). Alternatively, plate 50 may include slots 198 (FIG. 16) for receiving arm end 193 and arm end 194. In this embodiment, arm ends 193, 194 preferably have a shape that substantially corresponds to the shape of slots 198. While tensioner 170 is discussed herein with respect to FIGS. 11-17 as having two arms, it is to be understood that tensioner 170 may have more than two arms.

First arm 191 and second arm 192 are preferably connected to each other by arm connector 190 having hinge member 195 disposed between arm connector 190 and first arm 191 and second arm 192. Arm connector 190 includes hole 300 to permit shaft 180 to pass through arm connector 190 and to facilitate the movement of arm connector 190 and, thus, first arm 191 and second arm 192 along longitudinal axis 185 of shaft 180 in the directions of arrow 202 and arrow 203.

First end 181 includes handle 186 that facilitates shaft 180 to be rotated along shaft longitudinal axis 185 in the direction of arrows 202, 203 so that arm connector 190 and first arm 191 and second arm 192 are permitted to move along shaft 180 in the direction of arrows 202, 203.

Second end 182 includes base 188 that is connected to shaft 180. Preferably, base 188 is rotatably connected to shaft 180, such as through the use of bearings (not shown), ball joint 210 (FIG. 13), or base shaft 211 (FIG. 14) disposed perpendicular to base 188 and that extends into shaft cavity 212 in shaft 180, such that base 188 can engage plate 50, however, base 188 will not be rotated during activation of tensioner 170. In other words, base 188 remains stationarily engaged with plate 50 during the rotation of shaft 180 and, thus, the movement of plate 40 from the preformed shape to each of the at least one elastic shapes. In a preferred embodiment, base shaft 211 includes base shaft flange 214 and cavity 212 of shaft 180 include cavity flange 216 to prevent shaft 180 from easily being disconnected from base 188.

In the embodiment shown in FIG. 11, shaft 180 includes screw grooves 189 disposed along shaft 180 such that arm connector 190 is moved in the direction of arrow 202 so that first arm 191 and second arm 192 gradually move plate 50 from its pre-formed shape to at least one of its elastic shape as discussed above in greater detail. In this embodiment, hole 300 in arm connector 190 includes one or more corresponding screw grooves (not shown) to facilitate the movement of arm connector 190 and, thus, first arm 191 and second arm 192 along shaft 180 in the direction of arrows 202, 203. Additionally, a ratchet (not shown) may be used to facilitate turning shaft 180 in the direction of arrows 201, 202.

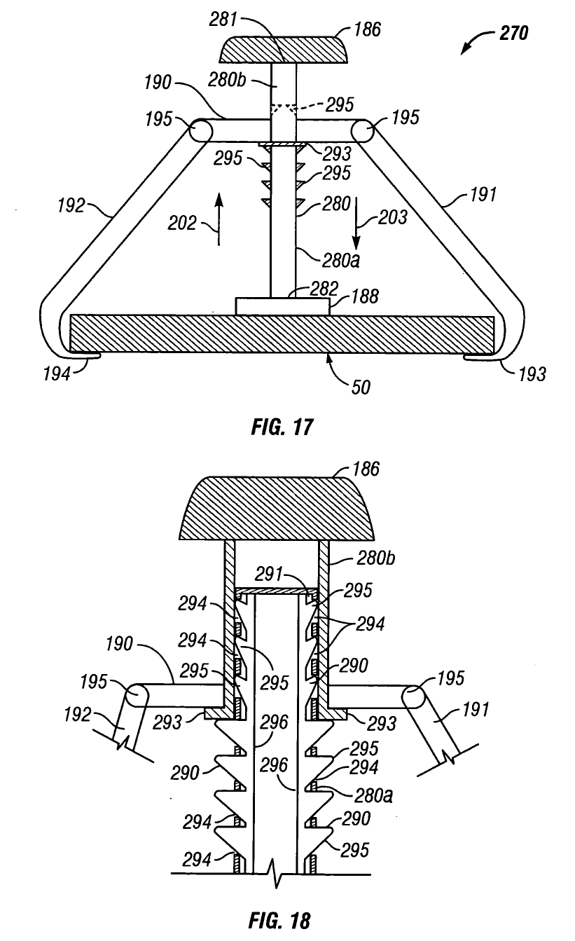

In another embodiment shown in FIGS. 17-18, shaft 280 includes inner shaft member 280a slidably engaged with outer shaft member 280b. In this embodiment, handle 186 is used to move outer shaft member 280b in the direction of arrows 202 and 203 by pushing or pulling handle in the direction of arrows 202 and 203. In a preferred embodiment, outer shaft member 280b includes shaft flange 293 for engaging arm connector 190 to facilitate movement of arm connector 190 in the direction of arrow 202.

Inner shaft member 280a and/or outer shaft member 280b includes at least one arm connector support member 290 that secures arm connector 190 in a certain position and prevents arm connector 190 from moving in the direction of arrow 202. For example, arm connector support member 290 may be a flange (not shown) that is releasably secured to shaft 180. Alternatively, arm connector support member 290 may be “one-way” grooves (not shown) disposed along either inner shaft member 280a or outer shaft member 280b that permit easy movement of arm connector 190 in the direction of arrow 202, but restrict movement in direction of arrow 203, i.e., only with the assistance of a tool or by placing inner shaft member 280a and/or outer shaft member 280b in a predetermined position can arm connector 190 be permitted to move in the direction of arrow 203, in the same manner as the operation of plastic cable ties which are known to persons of ordinary skill in the art.

In the embodiment shown in FIGS. 17-18, at least one arm connector support member 290 is moveable support member 295 that is held within cavity 291 of inner shaft member 280a by outer shaft member 280b. As outer shaft member 280b is moved in the direction of arrow 202, one or more moveable support members 295 are released from within cavity 291 and permitted to protrude through inner shaft apertures 294. In this arrangement, outer shaft member 280b or, preferably, shaft flange 293, prevents outer shaft member 280b from moving in the direction of arrow 203 until moveable support members 295 are re-disposed within cavity of inner shaft member 280a. Accordingly, movable support members 295 facilitate movement of arm connector 190 in the direction of arrow 202 so that first arm 191 and second arm 192 can gradually move plate 50 from its pre-formed shape to at least one of its elastic shape as discussed above in greater detail. To permit arm connector 190 to move in the direction of arrow 203, the operator need only push and hold each moveable support members 295 back into cavity 291 while moving outer shaft member 280b in the direction of arrow 203, until outer shaft member 280b sufficiently covers each inner shaft aperture 294, thereby maintaining each moveable support member 295 within cavity 291.

Moveable support member 295 is preferably connected to support member backbone 296 which maybe flexible plastic and pre-positioned within cavity 291 such that each moveable support member 295 retains sufficient stored energy (due to the curvature or elasticity of support member backbone 296) so that each moveable support member 295 will move from within cavity 291, through inner shaft aperture 294, and into position to prevent outer shaft member 280b from moving in the direction of arrow 203 as outer shaft member 280b is moved in the direction of arrow 202 exposing inner shaft apertures 294. In this embodiment, support member backbone 296 is preferably secured within cavity 291 such that it will not move out of its desired and necessary position such that moveable support member 295 will not function properly.

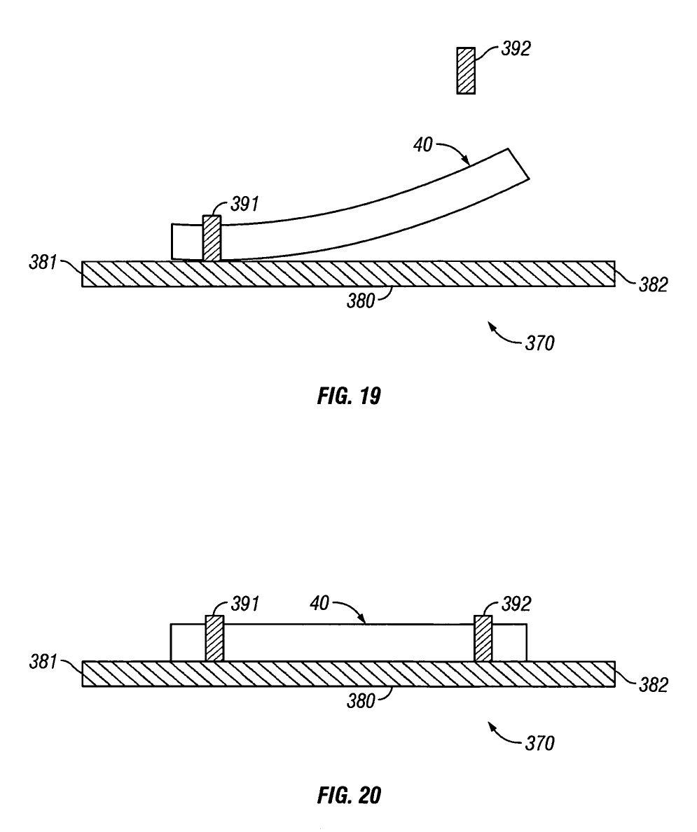

As shown in FIGS. 19-20, in still another embodiment, tensioner 370 comprises splint 380 having first end 381, second end 382, at least two fastening devices, wherein at least one of the fastening devices is removable such that the fastening devices may be releasably secured to splint 380. Bone compression device 40 is disposed along splint 380 by releasably securing bone compression device 40 to splint 380. In so doing, bone compression device 40 is moved from its pre-formed shape to at least one of the elastic shapes and releasably secured to splint 380. Therefore, if desired, bone compression device 40 may be “pre-packaged” ready for use at the manufacturing facilities.

Splint 380 includes first fastening device 391 and second fastening device 392. While both first fastening device 391 and second fastening device 392 may be removable, in the embodiment illustrated in FIG. 18, first fastening device 391 is not removable, i.e., it is affixed to splint 380, and second fastening device 392 is removable such that bone compression device 40 may be released from splint 380. Removable second fastening device 292 may be any removable fastening device known to persons skilled in the art. In this embodiment, bone compression device 40 is secured to splint 380 by placing bone compression device 40 underneath first fastening device 391. Bone compression device 40 is then moved from its pre-formed shape to at least one of the elastic shapes and releasably secured to splint 380 with second fastening device 392. It is to be understood that, in this specific embodiment, first fastening device 391 may be a clip, such as a staple that is secured to splint 380. Alternatively, first fastening device 391 maybe formed integral with splint 380.

In another aspect, the present invention is directed to methods of maintaining a bone in a spatial relationship, and in particular, at least two vertebrae in a spatial relationship with each other utilizing the bone compression devices discussed above. With respect to use of the bone compression devices for maintaining two or more vertebrae is spatial relationship with each other, first, bone compression device 40 is contoured by moving bone compression device 40 from its preformed shape to at least one of the at least one elastic shapes corresponding to the bone radius of curvature. The bone radius of curvature is formed by the at least two vertebrae. Bone compression device 40 is then disposed along the bone radius of curvature and secured to the at least two vertebrae.

This method of maintaining at least two vertebrae in a spatial relationship with each other may be used in connection with the insertion of at least one bone graft between the at least two vertebrae prior to securing the bone compression device to the at least two vertebrae. Accordingly, the bone compression devices facilitate maintaining the bone graft between the vertebrae as well as maintaining the at least two vertebrae in a spatial relationship with each other.

It is also noted that the bone compression devices may also be used for maintaining at least three, at least four, and at least five vertebrae in a spatial relationship with each other. Another feature of the method of maintaining at least two vertebrae in a spatial relationship with each other.

The methods of maintaining at least two vertebrae in a spatial relationship with each other may also include steps directed to order in which the fasteners are inserted through the fastener holes 58. For example, in one embodiment, bone compression device 40 includes four fastener holes 58 (FIGS. 4-7). The first fastener hole 58, 101 near first end 51 (upper left in FIGS.5 and 7) is disposed diagonally from fourth fastener hole 58, 104 near second end 52 (lower right in FIGS. 5 and 7) and second fastener hole 58, 102 near first end 51 (upper right in FIGS. 5 and 7) is disposed diagonally from third fastener hole 58, 103 near second end 52 (lower left in FIGS. 5 and 7). A first fastener is then inserted through first fastener hole 58, 101 and first end 51 of plate 50 is secured to the bone (one of the at least one vertebrae). A second fastener is then inserted through fourth fastener hole 58, 104 and second end 52 of plate 50 is secured to the bone (one of the at least one vertebrae). First end 51 of plate 50 is then further secured to the bone (one of the at least two vertebrae) with a third fastener inserted through second fastener hole 58, 102, and second end 52 of plate 50 is further secured to the bone (one of the at least two vertebrae) with a fourth fastener inserted through the third fastener hole 58, 103. It is believed that by inserting the fasteners through the fastener holes 58 to secure plate 50 to the bone in this manner, torsional resistence is lessened, thereby increasing the likelihood that bone compression device 40 will not be loosened over time.

It is to be understood that the invention is not limited to the exact details of construction, operation, exact materials, or embodiments shown and described, as obvious modifications and equivalents will be apparent to one skilled in the art. For example, while the bone compression device illustrated and described above, is discussed in connection with vertebrae, it may be used to with any other individual bone or bones. The dimensions and shapes, as well as the means for attaching the bone compression device to any bone, or number of bones, can be easily determined by a person of ordinary skill in the art. Moreover, while the bone compression devices have been described as being installed on the anterior side of the spine or other bone, the bone compression devices may be installed on the posterior side of the spine or other bone. Additionally, the bone compression devices may be installed on any vertebrae, i.e., lumbar, thoracic, cervical, or sacral. Further, the lower surface of the plate may include projections, or spikes, to facilitate securing the plate to the bone. Moreover, the plate interface member and corresponding plate interface may be any shape desired or necessary to permit the plate interface to securely capture the plate interface and thus permit manipulation and placement of the plate during installation of the plate. Additionally, the bone compression device may lack a distinguishable longitudinal axis, e.g., have a squared shape, or have the pre-formed, deformed, and elastic radii of curvature disposed along the width or lateral axis instead of the length or longitudinal axis. Further, it is to be understood that the plate may include a number of pre-formed radii of curvature, deformed radii of curvature, and elastic radii of curvature at any one time. Therefore, a single plate may be adapted to be secured to two or more different bone radii of curvature along one or more bones. Moreover, the tensioner may include more than two arms to facilitate the movement of the bone compression device from its pre-formed shape to one or more of its elastic shapes. Further, arm connector support member 290 may be disposed along, or connected to, either inner shaft member 280a or outer shaft member 280b. Accordingly, the invention is therefore to be limited only by the scope of the appended claims.

What is claimed:

1. A tensioner for facilitating the movement of a plate of a bone compression device, the plate having a pre-formed shape and at least one elastic shape that substantially corresponds to at least one bone radius of curvature of at least one bone, the tensioner comprising:
a shaft,
a base, and
at least two arms in communication with the shaft, the at least two arms adapted to be releasably secured to the plate,
the shaft having a shaft first end, a shaft second end, a shaft longitudinal axis, and at least one screw groove disposed along the shaft longitudinal axis to facilitate the movement of the at least two arms along the longitudinal axis of the shaft, the shaft second end being connected to the base.

2. The tensioner of claim 1, further comprising at least three arms adapted to be releasably secured to the plate.

3. The tensioner of claim 1, further comprising at least four arms adapted to be releasably secured to the plate.

4. The tensioner of claim 1, wherein at least two of the at least two arms of the tensioner include an arm end, the arm end having a shape that permits insertion of the arm end into a slot disposed along the plate.

5. The tensioner of claim 1, wherein at least two of the at least two arms of the tensioner include an arm end, the arm end having a hook.

6. The tensioner of claim 1, wherein at least two of the at least two arms of the tensioner include an arm end, the arm end having a spatula member.

7. The tensioner of claim 1, wherein at least two of the at least two arms of the tensioner are operatively associated with an arm connector by a hinge member.

8. The tensioner of claim 1, wherein the shaft second end is rotatably connected to the base such that the shaft is permitted to rotate and the base is permitted to remain stationary.

9. The tensioner of claim 1, wherein the shaft of the tensioner is rotatably connected to the base by a ball joint.

10. The tensioner of claim 1, wherein the shaft of the tensioner is rotatably connected to the base by a base shaft disposed within a cavity of the shaft.

11. A tensioner for facilitating the movement of a plate of a bone compression device, the plate having a pre-formed shape and at least one elastic shape that substantially corresponds to at least one bone radius of curvature of at least one bone, the tensioner comprising:
a splint having a first end and a second end and at least two fastening members, wherein at least one of the at least two fastening members is adapted to be releasably secured to the splint.

12. The tensioner of claim 11, wherein each of the at least two fastening members is adapted to be releasably secured to the splint.

13. A tensioner for facilitating the movement of a plate of a bone compression device, the plate having a pre-formed shape and at least one elastic shape that substantially corresponds to at least one bone radius of curvature of at least one bone, the tensioner comprising:
a shaft, a base, and
at least two arms in communication with the shaft, the at least two arms adapted to be releasably secured to the plate,
the shaft having a shaft first end, a shaft second end, a shaft longitudinal axis, an inner shaft member slidably engaged with an outer shaft member, and at least one arm connector support member disposed along the shaft longitudinal axis to maintain the at least two arms at a position along the longitudinal axis of the shaft, the shaft second end being connected to the base.

14. The tensioner of claim 13, wherein the shaft outer member includes a shaft flange.

15. The tensioner of claim 14, wherein each of the at least one arm connector support members are movable support members and the inner shaft member includes at least one inner shaft aperture corresponding to each of the at least one movable support members.

16. A bone compression device for placing in communication with at least one bone having at least one bone radius of curvature, the bone compression device comprising a plate having a preformed shape, the pre-formed shape having at least one pre-formed radius of curvature corresponding to each of the at least one bone radii of curvature, each of the at least one pre-formed radii of curvature being less than each of the corresponding at least one bone radii of curvature.

17. The bone compression device of claim 16, wherein the plate includes a deformed shape and at least one elastic shape between the pre-formed shape and the deformed shape, the deformed shape having a deformed radius of curvature greater than at least one of the bone radii of curvature, and at least one of the at least one elastic shapes having an elastic radius of curvature that substantially corresponds to at least one of the at least one bone radii of curvature.

18. The bone compression device of claim 17, wherein the plate includes a first end, a second end, and at least two attachment members.

19. The bone compression device of claim 18, wherein at least one of the at least two attachment members is a hole.

20. The bone compression device of claim 18, wherein at least one of the at least two attachment members is a loop.

21. The bone compression device of claim 18, wherein at least one of the two attachment members is disposed at the first end of the plate and another of the at least two attachment members is disposed at the second end of the plate.

22. The bone compression device of claim 21, wherein at least one of the at least two attachment members is a hole.

23. The bone compression device of claim 21, wherein at least one of the at least two attachment members is a loop.

24. The bone compression device of claim 16, wherein the plate includes a first end, a second end, and at least two attachment members.

25. The bone compression device of claim 16, wherein the plate includes a length having a longitudinal axis and a width having a lateral axis, at least one of the at least one pre-formed radii of curvature being disposed along a portion of the length.

26. The bone compression device of claim 16, wherein the plate includes a length having a longitudinal axis and a width having a lateral axis, at least one of the at least one pre-formed radii of curvature being disposed along a portion of the width.

27. The bone compression device of claim 16, wherein the plate includes a first end, a second end, at least two fastener holes disposed near the first end, and at least two fastener holes disposed near the second end.

28. A bone compression system for placing in communication with at least one bone having at least one bone radius of curvature, the bone compression system comprising:
a plate having a first end, a second end, a pre-formed shape, a deformed shape, and at least one elastic shape between the pre-formed shape and the deformed shape, the pre-formed shape having at least one pre-formed radius of curvature wherein at least one of the at least one pre-formed radii of curvature is less than at least one of the at least one bone radii of curvature, the deformed shape having a deformed radius of curvature greater than at least one of the at least one bone radii of curvature, and at least one of the at least one elastic shapes having an elastic radius of curvature that substantially corresponds to at least one of the at least one bone radii of curvature; and
a tensioner for facilitating the movement of the plate from the pre-formed shape to the at least one elastic shape that substantially corresponds to at least one of the at least one bone radii of curvature, the tensioner including a shaft, a base, and at least two arms adapted to be releasably secured to the plate, the shaft having a shaft first end, a shaft second end, a shaft longitudinal axis, and at least one screw groove disposed along the shaft longitudinal axis to facilitate the movement of the at least two arms along the longitudinal axis of the shaft, the shaft second end being connected to the base.

29. The bone compression system of claim 28, wherein the first and second attachment members of the plate are slots, the first slot being disposed at the first end of the plate and the second slot being disposed at the second end of the plate.

30. The bone compression system of claim 29, wherein at least two of the at least two arms of the tensioner include an arm end, the arm end having a shape that permits insertion of the arm ends into the first and second slots.

31. The bone compression system of claim 28, wherein the first and second attachment members of the plate are loops, the first loop being disposed at the first end of the plate and the second loop being disposed at the second end of the plate.

32. The bone compression system of claim 31, wherein at least two of the at least two arms of the tensioner include an arm end, the arm end having a hook that permits insertion of the arm end into the first and second loops.

33. The bone compression system of claim 28, wherein at least two of the at least two arms of the tensioner include an arm end, the arm end having a spatula member.

34. The bone compression system of claim 28, wherein at least two of the at least two arms of the tensioner are operatively associated with an arm connector by a hinge member.

35. The bone compression system of claim 28, wherein the shaft second end is rotatably connected to the base such that the shaft is permitted to rotate and the base is permitted to remain stationary.

36. The bone compression system of claim 35, wherein the shaft of the tensioner is rotatably connected to the base by a ball joint.

37. The bone compression system of claim 35, wherein the shaft of the tensioner is rotatably connected to the base by a base shaft disposed within a cavity of the shaft.

38. The bone compression system of claim 28, wherein the plate includes a length having a longitudinal axis and a width having a lateral axis, the pre-formed radius of curvature and the at least one elastic radius of curvature being disposed along the length.

39. The bone compression system of claim 28, wherein the plate includes a length having a longitudinal axis and a width having a lateral axis, the pre-formed radius of curvature and the at least one elastic radius of curvature being disposed along the width.
